(19) United States
(12) Patent Application Publication
(10) Pub. No.: US 2005/0069586 A1
(43) Pub. Date: Mar. 31, 2005

(54) Stable pharmaceutical compositions of 2-aza-bicyclo [3.3.0]-octane-3-carboxylic acid derivatives

(76) Inventors: Julia Hrakovsky, Rosh Ha-Ayin (IL); Ruth Tenengauzer, Hod Hasharon (IL)

Correspondence Address: KENYON & KENYON, ONE BROADWAY, NEW YORK, NY, 10004, US

(21) Appl. No.: 10/877,027

(22) Filed: Jun. 24, 2004

Related U.S. Application Data
(60) Provisional application No. 60/482,518, filed on Jun. 26, 2003.

(51) Int. Cl. A61K 31/407; A61K 9/20

(52) U.S. Cl. 424/464; 514/412

(57) ABSTRACT

A stable composition of a 2-aza-bicyclo[3.3.0]-octane-3-carboxylic acid derivative and a method for its preparation are described. The stable composition includes an intimate admixture of the derivative and a stabilizing effective amount of a lubricant. The stable composition further includes an external excipient. A method of preparing the stable composition includes forming an intimate admixture of the derivative and a lubricant and then blending the intimate admixture with at least one excipient. Preferably the final blend is transformed into solid unit dosage form.

### CROSS-REFERENCE TO RELATED APPLICATIONS

This application claims the benefit of provisional application Ser. No. 60/482,518, filed Jun. 26, 2003, which is incorporated herein by reference in its entirety.

### FIELD OF THE INVENTION

This invention relates to stabilized compositions of 2-aza-bicyclo[3.3.0]-octane-3-carboxylic acid derivatives and methods for preparing them.

### BACKGROUND OF THE INVENTION

Ramipril, quinapril, moexipril, fosinopril, enalapril, perindopril, and trandolapril are examples of 2-aza-bicyclo[3.3.0]-octane-3-carboxylic acid derivatives used in pharmaceutical formulations. Ramipril, which has the chemical name (2S, 3aS, 6aS)-1[(S)-N-[(S)-1-carboxy-3-phenyl-propyl] alanyl]octa hydrocyclopenta [b]pyrrole-2-carboxylic acid, 1-ethyl ester, is a pro-drug of ramiprilat, the active form of this angiotensin-converting enzyme (ACE) inhibitor.

Ramipril and certain other ACE inhibitors are reported to be effective antihypertensive drugs, but they are often susceptible to degradation. Ramipril is believed to degrade into two main products: diketopiperazine (DKP) and ramiprilat. Decomposition during manufacture and storage may adversely affect the effectiveness of the drug product or may cause the drug product to deviate from regulatory purity or potency requirements. It is therefore desirable to increase the stability of 2-aza-bicyclo[3.3.0]-octane-3-carboxylic acid derivative formulations.

The following chemical structures are some examples of 2-aza-bicyclo[3.3.0]-octane-3-carboxylic acid derivatives and their corresponding active form degradants.

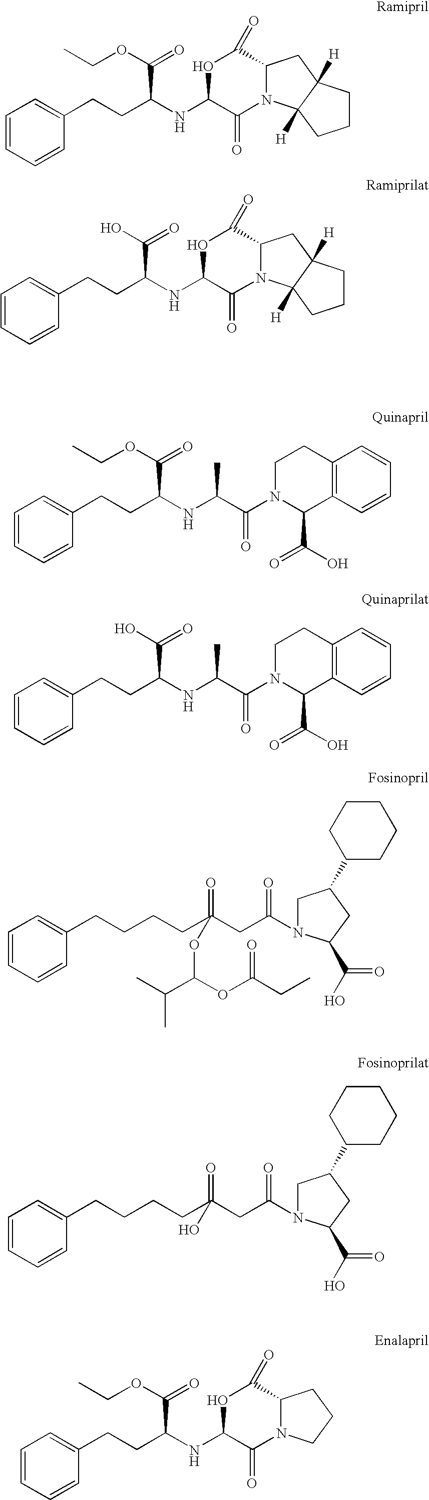

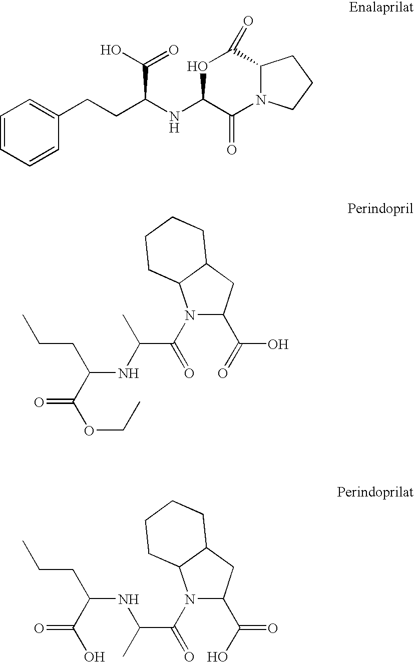

EP 280,999 B1 (Jan. 7, 1993) describes stabilized pharmaceutical compositions that contain ACE inhibitors, an alkali or alkaline earth metal carbonate, and a saccharide.

EP 317,878 B1 (Apr. 8, 1992) is directed towards stabilized compressed pharmaceutical formulations that may contain ramipril.

U.S. Pat. No. 6,417,196 is directed to ACE inhibitor-containing compositions stabilized by the presence of magnesium oxide.

U.S. Pat. No. 4,830,853 is directed towards the oxidation- and color-stability of certain ACE inhibitors.

U.S. Pat. No. 4,793,998 is directed towards minimizing cyclization and hydrolysis of certain ACE inhibitors.

### SUMMARY OF THE INVENTION

In one embodiment, the present invention provides a stable pharmaceutical composition comprising an intimate admixture including a 2-aza-bicyclo[3.3.0]-octane-3-carboxylic acid derivative and an effective amount of a lubricant to stabilize the composition, and at least one external excipient. In a preferred embodiment, the external excipient is in powder form.

In another embodiment, the present invention provides a method for preparing a stable pharmaceutical composition comprising forming an intimate admixture including an 2-aza-bicyclo[3.3.0]-octane-3-carboxylic acid derivative and an effective amount of a lubricant. The method further comprises blending the intimate admixture with an external excipient.

### DETAILED DESCRIPTION OF THE INVENTION

Definitions

As used herein, the term “stable pharmaceutical composition” refers to a pharmaceutical composition according to the invention that is less susceptible to degradation than a similar composition not having an intimate admixture of a derivative and a stabilizing effective amount of lubricant.

The term “effective amount” refers to possible weight percentages that will produce the intended effect of stabilizing the composition.

The term “by weight,” unless otherwise specified, means by weight of the total composition.

The term “by weight of the derivative” means by weight of the 2-aza-bicyclo[3.3.0]-octane-3-carboxylic acid derivative before degradation of the derivative.

The term “intimate admixture” refers to a mixture of closely-packed components, such as those exemplified herein, as opposed to a simple blend. An intimate admixture can be obtained, for example, by co-precipitation, co-milling, compression, granulation, or the like.

The term “external excipient” refers to an excipient or combination of excipients that have not been intimately admixed with a derivative.

The term “derivative” refers to a 2-aza-bicyclo[3.3.0]-octane-3-carboxylic acid derivative. 2-aza-bicyclo[3.3.0]-octane-3-carboxylic acid derivatives include, for example, ramipril, quinapril, moexipril, fosinopril, enalapril, perindopril, and trandolapril.

The term “principal degradant” refers to the single degradant from a 2-aza-bicyclo[3.3.0]-octane-3-carboxylic acid derivative with the highest percentage by weight. For example, the principal degradant of ramipril is usually diketopiperazine.

The term “active form degradant” refers to the active compound that 2-aza-bicyclo[3.3.0]-octane-3-carboxylic acid derivatives degrade into. For example, the active form degradant of ramipril is ramiprilat.

The term “DKP” refers to diketopiperazine.

In one embodiment, the present invention provides a stable pharmaceutical composition comprising (a) an intimate admixture including a 2-aza-bicyclo[3.3.0]-octane-3-carboxylic acid derivative and an effective amount of a lubricant to stabilize the composition, and (b) at least one excipient.

With respect to the intimate admixture of the stable pharmaceutical composition of the present invention, the derivative of the intimate admixture is preferably selected from the group consisting of ramipril, quinapril, moexipril, fosinopril, enalapril, perindopril, and trandolapril. The amount of the derivative is preferably from about 0.3% to about 6% by weight. More preferably, the derivative is present in the amount of from about 0.8% to about 5% by weight, and most preferably from about 0.8% to about 4.2% by weight.

The lubricant of the intimate admixture can be selected from the group consisting of magnesium stearate, talc, stearic acid, glycerylbehenate, polyethylene glycol, ethylene oxide polymers, sodium lauryl sulfate, magnesium lauryl sulfate, sodium oleate, sodium stearyl fumarate, DL-leucine, and others known in the art. Preferably, the lubricant is sodium stearyl fumarate. The effective amount of lubricant in the intimate admixture is preferably from about 0.3% to about 60% by weight, more preferably from about 0.8% to about 50% by weight, more preferably from about 1% to about 40% by weight, and more preferably from about 2% to about 10% by weight of the intimate admixture.

The intimate admixture can also include processing agents or other excipients that do not significantly adversely affect the stability of the composition. Thus, it is desirable to minimize the number and quantity of these additional excipients in the intimate admixture. In one embodiment, the intimate admixture further comprises one non-lubricant excipient. Preferably, the non-lubricant excipient is in the amount of about 95% by weight of the intimate admixture, or less, preferably less than about 50%.

Examples of possible excipients are spray-dried monohydrate lactose or anhydrous lactose, sucrose, dextrose, mannitol, sorbitol, starch, pregelatinized starch (e.g. starch 1500), cellulose (e.g. microcrystalline cellulose; Avicel), dihydrated or anhydrous dibasic calcium phosphate (available commercially under the registered trademark Emcompress from Mendell or A-Tab and Di-Tab from Rhone-Poulenc, Inc., Monmouth Junction, N.J.), calcium carbonate, calcium sulfate, and others as known in the art. To improve flowability, a preferred excipient is microcrystalline cellulose, preferably in the amount of about 30% or less by weight of the total composition.

The intimate admixture can also include disintegrants, binders, coloring agents, buffering agents, and other commonly employed pharmaceutically acceptable agents, provided they do not cause substantial degradation of the derivative, which is believed to be particularly sensitive to acidic agents.

With respect to the external excipient of the stable pharmaceutical composition of the present invention, the external excipient may include one or more excipients, such as processing agents. A preferred processing agent is microcrystalline cellulose. Preferably, the external excipient is in the amount of from about 20% to about 99% by weight, more preferably from about 40% to about 98% by weight, and more preferably from about 50% to about 90% by weight.

The external excipient may include, for example, a lubricant, such as those described herein. A preferred lubricant is sodium stearyl fumarate or magnesium hydroxide. Preferably, the amount of lubricant in the external excipient, if any, is in the amount of from about 0.3% to about 10% by weight, more preferably from about 0.5% to about 3% by weight, and more preferably from about 0.8% to about 2% by weight.

Alternatively or additionally, the external excipient may also include disintegrants, binders, coloring agents, buffering agents, and/or other commonly employed pharmaceutically acceptable agents.

Examples of suitable disintegrants are starch, pregelatinized starch, sodium starch glycolate, sodium carboxymethylcellulose, crosslinked sodium carboxymethylcellulose (e.g., sodium croscarmellose; crosslinked starch available under the registered trademark Ac-Di-Sol from FMC Corp., Philadelphia, Pa.), clays (e.g., magnesium aluminum silicate), microcrystalline cellulose (such as those available under the registered trademark Avicel from FMC Corp. or the registered trademark Emcocel from Mendell Corp., Carmel, N.Y.), alginates, gums, surfactants, effervescent mixtures, hydrous aluminum silicate, cross-linked polyvinylpyrrolidone (available commercially under the registered trademark PVP-XL from International Specialty Products, Inc.), and others as known in the art.

Examples of suitable binders include, e.g., acacia, cellulose derivatives (such as methylcellulose and carboxymethylcellulose, hydroxypropylmethylcellulose, hydroxypropylcellulose, hydroxyethylcellulose), gelatin, glucose, dextrose, xylitol, polymethacrylates, polyvinylpyrrolidone, starch paste, sucrose, sorbitol, pregelatinized starch, gum tragacanth, alginic acids and salts thereof such as sodium alginate, magnesium aluminum silicate, polyethylene glycol, guar gum, bentonites, and the like.

Coloring agents may include titanium dioxide and/or dyes suitable for food such as those known as FD & C dyes and natural coloring agents such as grape skin extract, beet red powder, beta carotene, annato, carmine, turmeric, paprika, and so forth.

Examples of possible buffering agents include tribasic sodium phosphate, anhydrous sodium carbonate, glycine, magnesium hydroxide, and the like.

In another embodiment, the stable pharmaceutical composition further includes a diuretic agent. Acceptable diuretic agents include high-ceiling diuretics, furosemide, bumetanide, ethacrynic acid, torsemide, muzolimide, azosemide, piretanide, tripamide, chlorothiazide, hydrochlorothiazide, chlorthalidone, indapamide, metozalone, cyclopenthiazide, xipamide, mefruside, dorzolamide, acetazolamide, methazolamide, ethoxzolamide, cyclothiazide, clopamide, dichlorphenamide, hydroflumethiazide, trichlormethiazide, polythiazide and benzothiazide. A preferred diuretic agent is hydrochlorothiazide. The preferred amount of the diuretic agent, when present, is from about 0.5% to about 40% by weight, more preferably from about 1% to about 30% by weight, and more preferably from about 2% to about 15% by weight.

Preferably, the stable pharmaceutical composition of the present invention resists degradation when stored under high stress conditions. For example, when stored at 55° C. for 48 hours, preferably (1) the total amount of the principal degradant and the active form degradant is about 3.3% by weight of the derivative, or less, more preferably about 1% by weight of the derivative, or less; (2) the amount of the principal degradant present is about 3% by weight of the derivative, or less, more preferably about 1% by weight of the derivative, or less; and/or (3) the amount of the active form degradant present is about 0.3% by weight of the derivative, or less, more preferably about 0.2% by weight of the derivative, or less.

For example, for the derivative ramipril, when stored at 55° C. for 48 hours, (1) the total amount of DKP and ramiprilat is preferably about 3.3% by weight of the ramipril, or less, more preferably about 1% by weight of the ramipril, or less; (2) the amount of DKP present is preferably about 3% by weight of the ramipril, or less, more preferably about 1% by weight of the ramipril, or less; and/or (3) the amount of ramiprilat present is preferably about 0.3% by weight of the ramipril, or less, more preferably about 0.2% by weight of the ramipril, or less.

A stable pharmaceutical composition of the derivatives can be prepared by, first, forming an intimate admixture comprising a derivative and a lubricant. Second, the intimate admixture can be blended with an external excipient to form a final blend that is preferably transformed into solid unit dosage form, such as a tablet or capsule.

Preferably, the intimate admixture is in granular form. Granules can be formed, for example, by dry granulation or wet granulation. Wet granulation techniques are known in the art and involve mixing the ingredients with a solvent, such as ethanol or isopropyl alcohol, and drying the mixture to obtain granules. Dry granulation can be performed, for example, by compaction or slugging. Compaction techniques are well known in the art and typically include the use of a roller compactor. Slugging is a common technique in the field and involves the use of a tableting machine to produce slugs and passing the slugs through a mill or an oscillating granulator to form granules. Typical screen aperture sizes are, for example, 0.5 mm, 0.8 mm, or 1.0 mm.

The stable pharmaceutical composition of the present invention is preferably in solid unit dosage form, more preferably in tablet or capsule form. Conventional tableting processes can be employed, e.g., by forming a tablet from a desired mixture of ingredients into the appropriate shape using a conventional tablet press. Tablet formulation and processing techniques are generally known in the field. Capsule formulation methods are also commonly known in the art.

The functions and advantages of these and other embodiments of the present invention will be more fully understood from the examples below. The following examples are intended to illustrate the benefits of the present invention, but do not exemplify the full scope of the invention.

### EXAMPLES

### Examples 1-2

Dry Granulation

The following tablets were prepared by dry granulation. The ingredients of Part I of Table 1 were blended, and initial compression was carried out using a rotary tableting machine. The compressed material was milled through an oscillating granulator to produce granules having an average diameter of about 0.8 mm. The external excipient was added (Part II) and the final blend was then compressed to form tablets.

TABLE 1

| | Example 1 | Example 2 |
|---|---|---|
| Ingredient | mg/tablet | mg/tablet |
| Part I | | |
| Ramipril | 1.25 | 1.25 |
| Microcrystalline Cellulose | 20.0 | — |
| Sodium Stearyl Fumarate | 0.5 | 1.0 |
| Part II | | |
| Microcrystalline Cellulose | 90.5 | 110.5 |
| Magnesium Hydroxide | 6.25 | 6.25 |
| Sodium Stearyl Fumarate | 1.5 | 1.0 |

### Comparative Examples 3-5

Direct Compression

The ingredients in Table 2 were blended and compressed into tablets.

TABLE 2

| | Example 3 | Example 4 | Example 5 |
|---|---|---|---|
| Ingredient | mg/tablet | mg/tablet | mg/tablet |
| Ramipril | 1.25 | 1.25 | 1.25 |
| Microcrystalline | 115.3 | 112.75 | 100.75 |
| Cellulose | | | |
| Methocel E-5 ™ | — | 4.0 | — |
| Povidone (PVP K-30) | — | — | 3.0 |
| Magnesium Hydroxide | 6.25 | — | — |
| Colorant | 0.2 | — | — |
| Crospovidone | — | — | 13.0 |
| Sodium Stearyl | 2.0 | 2.0 | 2.0 |
| Fumarate | | | |

### Comparative Examples 6-7

Wet Granulation

The ingredients of Part I of Table 3 were granulated using isopropyl alcohol as a granulation liquid. The granulate was dried, milled and blended with ingredients from Part II. The final blend was compressed into tablets.

TABLE 3

| | Example 6 | Example 7 |
|---|---|---|
| Ingredient | mg/tablet | mg/tablet |
| Part I | | |
| Ramipril | 1.25 | 1.25 |
| Microcrystalline Cellulose | 30.0 | 30.0 |
| Isopropyl Alcohol | | |
| Part II | | |
| Microcrystalline Cellulose | 80.5 | 73.75 |
| Magnesium Hydroxide | 6.25 | — |
| Crospovidone | — | 13.0 |
| Sodium Stearyl Fumarate | 2.0 | 2.0 |

Results

A stability test was performed on each sample by packing the tablets in plastic containers and storing them in the oven at 55° C. with added water. After 48-hour storage, the amount of ramiprilat and DKP present were measured. High performance liquid chromatography (HPLC) was employed with the following parameters:
Column: Zorbax SB C-8, 5 μm, 250×4.6 mm
Mobile Phase: Buffer adjusted to pH 2.00 with acetonitrile (65:35 VN)
Flow Rate: 1.0 mL/min
Detection: UV, λ=215 nm
Column Temp.: 60° C.
Sample Temp.: 4° C.
Injection Volume: 50 μl

A stability test was also conducted for the marketed product Tritace® 1.25 mg, which is reported to contain ramipril, starch, microcrystalline cellulose, sodium stearyl fumarate, hypromellose, and colorant.

The results are shown in Table 4.

TABLE 4

| | Ramiprilat (%) | DKP (%) after | Total % of DKP and |
|---|---|---|---|
| Example No. | after 48 h at 55° C. | 48 h at 55° C. | Ramiprilat |
| 1 | 0.26 | 2.92 | 3.18 |
| 2 | 0.16 | 0.80 | 0.96 |
| 3 | 0.26 | 8.23 | 8.49 |
| 4 | 0.08 | 18.65 | 18.73 |
| 5 | 0.06 | 15.45 | 15.51 |
| 6 | 0.13 | 11.81 | 11.94 |
| 7 | 0.08 | 14.80 | 14.88 |
| Tritace ® | 0.06 | 1.36 | 1.42 |

1. A stable pharmaceutical composition comprising:
an intimate admixture including a 2-aza-bicyclo[3.3.0]-octane-3-carboxylic acid derivative and an effective amount of a lubricant to stabilize the composition; and
an external excipient.

2. The composition according to claim 1, wherein the intimate admixture is in granular form.

3. The composition according to claim 1, wherein the effective amount of the lubricant ranges from about 0.3% to about 60% by weight of the intimate admixture.

4. The composition according to claim 1, wherein the effective amount of the lubricant ranges from about 0.8% to about 50% by weight of the intimate admixture.

5. The composition according to claim 1, wherein the effective amount of the lubricant ranges from about 1% to about 40% by weight of the intimate admixture.

6. The composition according to claim 1, wherein the effective amount of the lubricant ranges from about 2% to about 10% by weight of the intimate admixture.

7. The composition according to claim 1, wherein the lubricant is selected from the group consisting of magnesium stearate, talc, stearic acid, glycerylbehenate, polyethylene glycol, ethylene oxide polymers, sodium lauryl sulfate, magnesium lauryl sulfate, sodium oleate, DL-leucine, and sodium stearyl fumarate.

8. The composition according to claim 7, wherein the lubricant is sodium stearyl fumarate.

9. The composition according to claim 1, wherein the intimate admixture further comprises one non-lubricant excipient.

10. The composition according to claim 9, wherein the non-lubricant excipient is microcrystalline cellulose.

11. The composition according to claim 9, wherein the non-lubricant excipient is in the amount of about 95% or less by weight of the intimate admixture.

12. The composition according to claim 1, further comprising a diuretic agent.

13. The composition according to claim 12, wherein the diuretic agent is hydrochlorothiazide.

14. The composition according to claim 1, wherein the derivative is selected from the group consisting of ramipril, quinapril, moexipril, fosinopril, enalapril, perindopril, and trandolapril.

15. The composition according to claim 14, wherein the derivative is ramipril.

16. The composition according to claim 1, wherein the derivative is present in an amount of from about 0.3% to about 6% by weight of the total composition.

17. The composition according to claim 1, wherein the derivative is present in an amount of from about 0.8% to about 5% by weight of the total composition.

18. The composition according to claim 1, wherein the derivative is present in an amount of from about 0.8% to about 4.2% by weight of the total composition.

19. The composition according to claim 1, wherein the composition is in solid unit dosage form.

20. The composition according to claim 19, wherein the composition is in tablet form.

21. The composition according to claim 19, wherein the composition is in capsule form.

22. The composition according to claim 1, wherein the amount of a principal degradant present in the composition after 48 hours at 55° C. is less than 3% by weight of the derivative.

23. The composition according to claim 1, wherein the amount of a principal degradant present in the composition after 48 hours at 55° C. is less than 1% by weight of the derivative.

24. The composition according to claim 22, wherein the principal degradant is diketopiperazine and the derivative is rampiril.

25. The composition according to claim 23, wherein the principal degradant is diketopiperazine and the derivative is rampiril.

26. The composition according to claim 1, wherein the amount of an active form degradant present in the composition after 48 hours at 55° C. is less than 0.3% by weight of the derivative.

27. The composition according to claim 1, wherein the amount of an active form degradant present in the composition after 48 hours at 55° C. is less than 0.2% by weight of the derivative.

28. The composition according to claim 1, wherein the total amount of an active form degradant and a principal degradant present in the composition after 48 hours at 55° C. is less than 3.3% by weight of the derivative.

29. The composition according to claim 1, wherein the total amount of an active form degradant and a principal degradant present in the composition after 48 hours at 55° C. is less than 1% by weight of the derivative.

30. The composition according to claim 28, wherein the active form degradant is ramprilat, the principal degradant is diketopiperazine, and the derivative is rampiril.

31. The composition according to claim 29, wherein the active form degradant is ramprilat, the principal degradant is diketopiperazine, and the derivative is rampiril.

32. A method for preparing a stable pharmaceutical composition comprising:
forming an intimate admixture including a 2-aza-bicyclo[3.3.0]-octane-3-carboxylic acid derivative and a lubricant; and
blending the intimate admixture with an external excipient.

33. The method of claim 32, further comprising transforming the final blend into solid unit dosage form.

34. The method of claim 33, wherein the composition is in tablet form.

35. The method of claim 33, wherein the composition is in capsule form.

36. The method of claim 32, wherein the intimate admixture is in granular form.

37. The method of claim 32, wherein the intimate admixture is formed by dry granulation or wet granulation.

38. A 2-aza-bicyclo[3.3.0]-octane-3-carboxylic acid derivative composition made by the process of claim 32.
